(19) United States
(12) Patent Application Publication
(10) Pub. No.: US 2023/0071939 A1
(43) Pub. Date: Mar. 9, 2023

(54) APPARATUS AND METHODS FOR DELIVERY OF CELL SUSPENSION

(71) Applicant: FUJIFILM Cellular Dynamics, Inc., Madison, WI (US)

(72) Inventors: Randall D. LEARISH, Madison, WI (US); Nathaniel A. BEARDSLEY, Madison, WI (US); Sho SATO, Madison, WI (US); Christopher W. McMAHON, Madison, WI (US)

(73) Assignee: FUJIFILM Cellular Dynamics, Inc., Madison, WI (US)

(21) Appl. No.: 17/760,228

(22) PCT Filed: Feb. 2, 2021
(86) PCT No.: PCT/US2021/016151

§ 371 Date: Aug. 5, 2022

Related U.S. Application Data
(60) Provisional application No. 62/970,357, filed on Feb. 5, 2020.

(51) Int. Cl. A61M 5/145

(52) U.S. Cl. CPC A61M 5/1456; A61M 2205/3327; A61M 2210/0687; A61M 2206/20

(57) ABSTRACT

Apparatus and methods for monitoring and delivering a cell suspension. In certain examples, the apparatus comprises a reservoir containing the cell suspension, a cannula, and a liquid transfer mechanism configured to transfer the cell suspension from the reservoir to the cannula. The apparatus can also comprise an agitation mechanism to agitate the cell suspension, and a monitoring device to quantify a concentration of cells transferred from the reservoir to the cannula.

### CROSS-REFERENCE TO RELATED APPLICATIONS

This application claims the benefit of United States Provisional Patent Application Nos. 62/970,357, filed Feb. 5, 2020, the entirety of which is incorporated herein by reference.

### BACKGROUND INFORMATION

Apparatus and methods to control cell delivery into a patient are needed for clinical cell therapies. Certain therapeutic applications include the delivery of Good Manufacturing Practice (GMP)-manufactured dopaminergic (DA) neurons to the brains of Parkinson's patients. Particular applications, for example, may include thawing a bank of cryopreserved cells, re-suspending them in a physiologic saline solution at a high cell concentration, and then injecting small volumes (doses) into a specific region of the midbrain. In such applications, a stereotaxic frame can be attached to the patient's head and a narrow neuro-cannula guided repeatedly through holes in the skull. The neuro-cannula can be re-positioned numerous times on both left and right hemispheres of the brain so that multiple doses of cells can be spread across the putamen. There is precedence for this type of cell delivery approach using fetal tissue-derived cells (RA Barker, Lancet Neurol. 2013 January; 12(1) 84-91).

However, there can be significant “dosing” problems in this endeavor. Safety concerns limit the dosing volumes that can be injected into the brain. The Parkinson's application, for example, proposes to deliver 20 μl per needle tract. The number of cells to achieve efficacy is predicted to be 0.3-0.9×10⁶ cells per tract. Thus, the cell product must be prepared at a relatively high concentration (0.9×10⁶/0.02 ml=45×10⁶/ml). Unlike the delivery of a soluble injectable drug compound which is easily transferred from its product container (e.g., a glass vial) to a syringe and then to the patient, such high concentrations of cells are more difficult to handle because they do not stay in suspension in standard physiologic saline solutions.

Thus, when cells are prepared using these preferred solutions (e.g. BSS Plus® solution), and held in a syringe or tube, they can undergo a rapid phase separation and settle out of solution. This can lead to unacceptably high variability in the concentrations of cells (doses) delivered from the syringe. For neurosurgery involving incremental delivery of small volumes, this phenomenon can translate into either too many cells (over-dosing) or too few cells (under-dosing) at each site of delivery.

For a device to be useful for a cell therapy dosing strategy, it needs to do more than specify accurate volumes. It should also specify the accurate delivery of a pre-determined number of cells. Limited information is known about what if any attempts were made to control for this dosing problem and to specify the number of cells delivered in previous Parkinson's cell therapy trials. However, there is a growing awareness that this problem needs to be addressed (MH Amer, et. al. Nature Partner Journals Regenerative Medicine, (2017)).

Accordingly, an effective cell therapy approach (e.g. including for Parkinson's disease) needs a transfer mechanism that is better suited for timed delivery from small volume syringes. In addition, a better method of delivering highly concentrated cell suspensions through medical tubing is also desired. Furthermore, a means of monitoring the transfer of cells from a syringe into medical tubing (or other conduit) is also desirable.

Accordingly, a need exists for accurately monitoring and controlling cell delivery for effective clinical cell therapies.

### SUMMARY

As explained in more detail below, exemplary embodiments of the present disclosure enable improvements in many aspects of cell delivery for clinical cell therapies.

Certain embodiments include an apparatus for monitoring and delivering a cell suspension, where the apparatus comprises: a reservoir containing the cell suspension; a cannula in fluid communication with the reservoir; a liquid transfer mechanism configured to transfer the cell suspension from the reservoir to the cannula; an agitation mechanism configured to agitate the cell suspension; and a monitoring device configured to quantify a concentration of cells transferred from the reservoir to the cannula.

In particular embodiments, the monitoring device records a digital image of the cell suspension. In some embodiments, the digital image is a still image and in specific embodiments the digital image is a moving image.

In particular embodiments, the monitoring device is configured to quantify a number of cells transferred to the cannula. In some embodiments, the monitoring device is an optical measurement device. In specific embodiments, the optical measurement device is configured to detect an amount of light transmitted through the cell suspension. In certain embodiments, the optical measurement device is configured to detect an amount of light transmitted reflected by the cell suspension. In particular embodiments, the monitoring device is configured to measure the turbidity of the cell suspension. In some embodiments, the liquid transfer mechanism comprises a syringe and in particular embodiments the syringe is a threaded syringe. In specific embodiments, the liquid transfer mechanism comprises a syringe pump. In certain embodiments, the liquid transfer mechanism comprises a pump. In particular embodiments, the pump is a peristaltic pump. In some embodiments, the liquid transfer mechanism comprises an inflatable bladder.

In specific embodiments, the apparatus further comprises a conduit in fluid communication with the reservoir and the cannula. In certain embodiments, the conduit comprises a proximal end and a distal end; the reservoir is coupled to the proximal end of the conduit; and the cannula is coupled to the distal end of the conduit. In particular embodiments, the agitation mechanism is configured to impart motion to the reservoir. Some embodiments, further comprise a mixing element in the reservoir. In specific embodiments, the mixing element is a bubble. In certain embodiments, the mixing element is a spherical ball bearing.

Particular embodiments include an apparatus for monitoring and delivering a cell suspension, where the apparatus comprises: a first reservoir containing a liquid; a second reservoir containing a cell suspension; an agitation mechanism configured to agitate the cell suspension; a conduit in fluid communication with the first reservoir and the second reservoir; a cannula in fluid communication with the conduit and the second reservoir; and a liquid transfer mechanism configured to transfer the cell suspension from the second reservoir to the cannula. In some embodiments, the monitoring device is configured to quantify a number of cells transferred to the cannula. In specific embodiments, the liquid transfer mechanism is configured to move in a first direction to transfer the cell suspension from the second reservoir to the cannula; and the liquid transfer mechanism is configured to move in a second direction to transfer the cell suspension through the cannula.

In certain embodiments, the conduit comprises a proximal end and a distal end; the first reservoir is coupled to the proximal end of the conduit; and the second reservoir is coupled to the distal end of the conduit. In particular embodiments, the monitoring device is an optical measurement device. In some embodiments, the optical measurement device is configured to detect an amount of light transmitted through the cell suspension. In specific embodiments, the optical measurement device is configured to detect an amount of light transmitted reflected by the cell suspension. In certain embodiments, the monitoring device is configured to measure the turbidity of the cell suspension. In particular embodiments, the liquid transfer mechanism comprises a syringe, and in certain embodiments the syringe is a threaded syringe. In some embodiments, the liquid transfer mechanism comprises a syringe pump. In specific embodiments, the liquid transfer mechanism comprises a pump. In certain embodiments, the pump is a peristaltic pump. In particular embodiments, the liquid transfer mechanism comprises an inflatable bladder.

In some embodiments, the apparatus further comprises a conduit in fluid communication with the reservoir and the cannula. In specific embodiments, the conduit comprises a proximal end and a distal end; the reservoir is coupled to the proximal end of the conduit; and the cannula is coupled to the distal end of the conduit. In particular embodiments, the agitation mechanism is configured to impart motion to the reservoir. Some embodiments, further comprise a mixing element in the reservoir. In specific embodiments, the mixing element is a bubble. In certain embodiments, the mixing element comprises one or more spherical ball bearings.

Particular embodiments include a method for monitoring and delivering a cell suspension, where the method comprises: providing a reservoir containing the cell suspension; providing a cannula in fluid communication with the reservoir; agitating the cell suspension; transferring the cell suspension from the reservoir to the cannula; and monitoring a concentration of cells in the cell suspension transferred from the reservoir to the cannula. In some embodiments, transferring the cell suspension from the reservoir to the cannula comprises transferring the cell suspension through a conduit. In specific embodiments, monitoring the concentration of cells transferred from the reservoir to the cannula comprises measuring an optical quality of the cell suspension. In certain embodiments, the reservoir containing the cell suspension is a first reservoir; and the method further comprises: transferring a liquid from a second reservoir to the cannula; and diluting the cell suspension with the liquid.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “about”, “substantially” and “approximately” mean, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises.” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

The following drawings form part of the present specification and are included to further demonstrate certain aspects of the present invention. The present disclosure may be better understood by reference to one or more of these drawings in combination with the detailed description of specific embodiments presented herein.

FIG. 1 displays a schematic view of an apparatus according to an exemplary embodiment of the present disclosure.

FIG. 2 displays a schematic view of an apparatus according to an exemplary embodiment of the present disclosure.

FIG. 3 displays a schematic view of an apparatus according to an exemplary embodiment of the present disclosure.

FIG. 4 displays an overview of steps that can be used in a method for monitoring and delivering a cell suspension according to an exemplary embodiment.

FIG. 5a-b displays an exemplary embodiment of the present disclosure. FIG. 5a is a photograph of medical tubing connected to a disposable syringe; an embodiment of the conduit 130 and reservoir 110 in FIG. 1, respectively. FIG. 5b is a photograph of the syringe and tubing mounted on to a syringe pump; an embodiment of the apparatus 195 in FIG. 1.

FIG. 6a-b displays an alternate embodiment of the present disclosure; FIG. 6a is a photograph of medical tubing connected to a Hamilton syringe; the tubing and syringe are represented as conduit 230 and reservoir 215 in FIG. 2, respectively. FIG. 6b is a photograph of the syringe and tubing mounted on to a reversible syringe pump; an embodiment of the apparatus 295 in FIG. 2.

FIG. 7a displays a photograph of the monitoring device represented as 160, 260, and 360 in FIGS. 1, 2, and 3 respectively.

FIG. 7b displays a photograph taken with the monitoring device of FIG. 7.

FIGS. 8a and 8b displays a photograph of an apparatus according to an exemplary embodiment of the present disclosure represented in FIG. 2.

FIGS. 9a and 9b displays a photograph of an apparatus used to obtain video and photographs such as those of FIG. 7b.

FIG. 10 displays a photograph of an apparatus according to an exemplary embodiment of the present disclosure represented in FIG. 2 with a second port that can be used to bleed out air bubbles.

### DETAILED DESCRIPTION OF ILLUSTRATIVE EMBODIMENTS

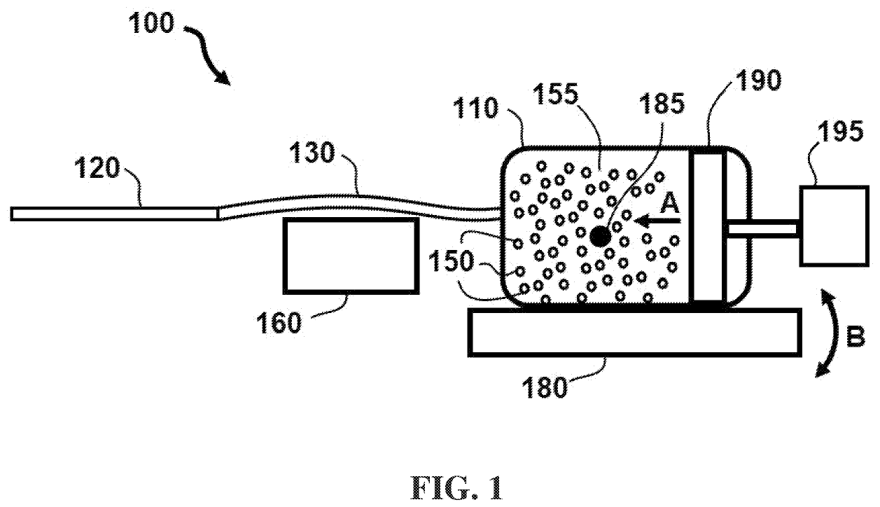

Referring initially to FIG. 1, a schematic of an apparatus 100 for monitoring and delivering a cell suspension is shown. In this embodiment, apparatus 100 comprises a reservoir 110 containing cells 150 in a suspension medium 155. In addition, apparatus 100 comprises a cannula 120 in fluid communication with reservoir 110. While the illustrated embodiment comprises a conduit 130 in fluid communication with both reservoir 110 and cannula 120, it is understood than other embodiments may not comprise conduit 130. For example, in other embodiments cannula 120 may be directly coupled to reservoir 110 without a conduit.

In the embodiment shown, apparatus 100 further comprises a liquid transfer mechanism 190 configured to transfer cells 150 and suspension medium 155 from reservoir 110 to cannula 120. In the embodiment shown, liquid transfer mechanism 190 is configured as a plunger disposed within reservoir 110. In specific embodiments, liquid transfer mechanism 190 may be a syringe, including for example, a syringe actuated by a syringe pump 195.

It is understood that in other embodiments, liquid transfer mechanism 190 may be configured as other devices capable of transferring liquid from reservoir 110. For example, liquid transfer mechanism 190 may be configured as an inflatable bladder disposed within reservoir 110 that displaces or transfers cells 150 and suspension medium 155 from reservoir 110 to cannula 120 as the bladder is inflated. In still other embodiments, liquid transfer mechanism 190 may be configured as a pump (including for example, a peristaltic pump or other type of pump that is external to reservoir 110).

In the illustrated embodiment, apparatus 100 further comprises an agitation mechanism 180 configured to agitate or provide turbulence to cells 150 and suspension medium 155. In exemplary embodiments, agitation mechanism 180 can impart motion to reservoir 110 to agitate suspension medium 155 (e.g. to ensure that cells are maintained in suspension within reservoir 110). In particular embodiments, agitation mechanism 180 may impart a rocking motion in the direction indicated by arrow B. In certain embodiments, agitation mechanism 180 may impart a vibrating, reciprocating or rotating motion to reservoir 110.

In some embodiments, apparatus 100 may also comprise a mixing element 185 configured to assist in suspending cells 150 in suspension medium 155. For example, mixing element 185 may be a bubble or solid element (e.g. a spherical ball bearing) that moves within reservoir 110 when agitation mechanism 180 imparts motion to reservoir 110. The movement of mixing element 185 within suspension medium 155 can prevent cells from settling out of suspension by mixing suspension medium 155 within reservoir 110. In some embodiments, agitation mechanism 180 may be configured as a magnet or electromagnet acting in conjunction with a mixing element 185 configured as one or more ball bearings. As used herein the term ball bearing includes any spherical element, which could be made of metal, magnetic, plastic or other materials, including coated materials (e.g. metal or magnet coated in plastic).

In addition, apparatus 100 also includes a monitoring device 160 configured to monitor, record and/or quantify the concentration of cells transferred from reservoir 110 to cannula 120.

In certain embodiments, monitoring device 160 may comprise a sensor that can detect a parameter that can be correlated to the concentration of cells. For example, monitoring device 160 may comprise an optical sensor that detects an amount of light transmitted through (or reflected or absorbed by) cell suspension 155 to determine the concentration of cells 150. In other embodiments, monitoring device 160 may comprise a flow sensor capable of measuring the turbidity of cell suspension 150 or detecting macromolecules, including for example, sensors available from Pendotech©. Monitoring device 160 may also be configured (e.g. via a computer processor and a computer readable medium) to quantify the number of cells moving transferred to cannula 120. In certain embodiments, sensor 160 may comprise a camera configured to record images including still or moving images (e.g. digital photographs or videos).

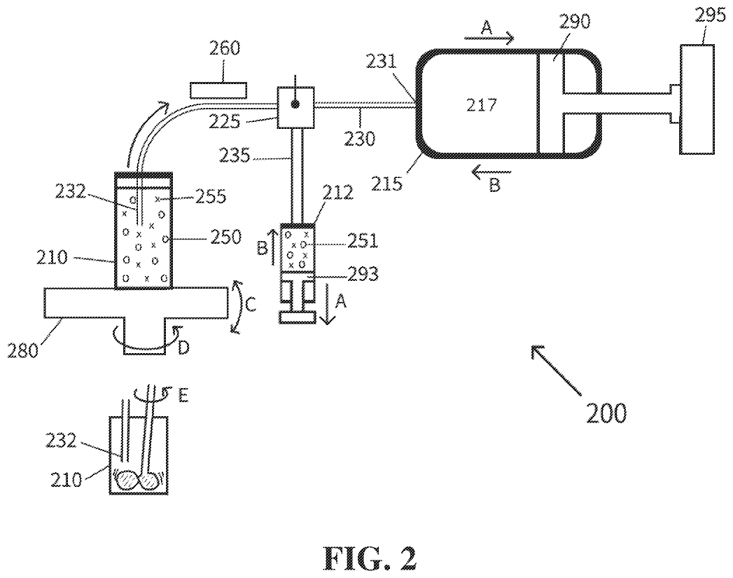

Referring now to FIG. 2, a schematic of another embodiment of an apparatus 200 for monitoring and delivering a cell suspension is shown. This embodiment is similar to the previously-described embodiment of FIG. 1, but includes a reservoir with a cell suspension that is distal from the liquid transfer mechanism and proximal to the cannula used to inject the cells.

In the embodiment shown in FIG. 2, apparatus 215 that contains a liquid 217 (e.g. a saline solution or other suitable liquid for cell injection). Apparatus 200 further comprises a second reservoir 210 containing cells 250 in a suspension medium 255 and an agitation mechanism 280 configured to agitate suspension medium 255. In addition, apparatus 200 comprises a conduit 230 in fluid communication with first reservoir 215 and second reservoir 210, as well as a cannula 232 in fluid communication with conduit 230 and second reservoir 210. In the embodiment shown, first reservoir 215 is coupled to a first proximal end 231 of conduit 230, while second reservoir 210 is coupled to a second distal end of cannula 232 of conduit 230.

In the embodiment shown in FIG. 2, apparatus 200 also comprises a liquid transfer mechanism 290 configured to transfer cells 250 in suspension medium 255 from second reservoir 210 to cannula 232. In a specific embodiment, liquid transfer mechanism 290 can be configured as a plunger (e.g. as part of a syringe) that can move in the direction of arrow A to transfer cells 250 in suspension medium 255 from second reservoir 210 to cannula 232. For example, when liquid transfer mechanism 290 is moved in the direction of arrow A, a vacuum can be created in conduit 230 and cannula 232, drawing cells 250 in suspension medium 255 from second reservoir 210 to cannula 232. After cells 250 in suspension medium 255 is drawn into the distal end of the cannula 232 and the conduit 230, the distal end of the cannula 232 can be moved to the subject patient (not shown) and the liquid transfer mechanism 290 can be moved in the direction of arrow B to transfer liquid 217 through conduit 230 and transfer cells 250 in suspension medium 255 and into the subject patient (not shown).

Similar to the previously described embodiment, liquid transfer mechanism 290 may be arranged in different configurations. In exemplary embodiments, liquid transfer mechanism 290 can be configured as any device capable of transferring liquid 217 from first reservoir 215 and cell suspension from second reservoir 210. In one specific embodiment, liquid transfer mechanism 290 may be configured as a syringe actuated by a syringe pump 295.

In a separate embodiment shown in FIG. 2, apparatus 200 may also include a third reservoir 212 that is coupled to the conduit 230 by a conduit 235 and a three-position or four-position valve 225 that allows flow to and from the third reservoir 212. The opening of the valve 225 provides fluid communication between reservoir 212 and conduit 230, and an alternative entry point to transfer cell suspension 250 to the conduit 230. In this embodiment, alternate opening and closing of the valve 225 allows alternating or sequential forward movement (in the direction of B) of the cell suspension 250 from reservoir 212 into the conduit 230 followed by forward movement (in the direction of B) of the liquid 217.

In one specific embodiment, liquid transfer mechanism 212 may be configured as a syringe actuated by a syringe pump 293 that is complementary to 295. Reverse and forward (A and B) movement of liquid transfer mechanisms 293 and 295 enables directional fluid transfer between reservoirs.

Apparatus 200 also comprises an agitation mechanism 280 configured to agitate cell suspension 250. Similar to the previously described embodiment, agitation mechanism 280 can impart motion to second reservoir 210 to agitate cell suspension 250 so that cells are maintained in suspension within second reservoir 210. In particular embodiments, agitation mechanism 280 may impart a rocking motion in the direction indicated by arrow C. In certain embodiments, agitation mechanism 280 may impart a vibrating, reciprocating or rotating motion to reservoir 210 indicated by arrow D. In a separate embodiment, motion to reservoir 210 may be imparted by a stirring rod or stick attached to a rotating motor indicated by arrow E.

It is understood that embodiments that impart agitation or motion are intended to assist in suspending cells and are applicable to cells that are located in any of the reservoirs 210, 215, or 212, or any combination of locations. Not all combinations are shown in the figures.

Cells 250 in suspension medium 255 can be at a higher concentration of cells than desired for delivery to the patient, so that cell suspension 250 and first liquid 217 are merged (e.g. in cannula 232) to obtain the desired concentration. Apparatus 200 can also include a monitoring device 260 configured to quantify the concentration of cells after filling the cannula in reverse (in the direction of A) from reservoir 210, or in the direction of B from reservoir 212 after cell suspension 251 is diluted with first liquid 217. In exemplary embodiments, monitoring device 260 may be configured and operate in a manner equivalent to monitoring device 160 discussed in the embodiment shown in FIG. 1.

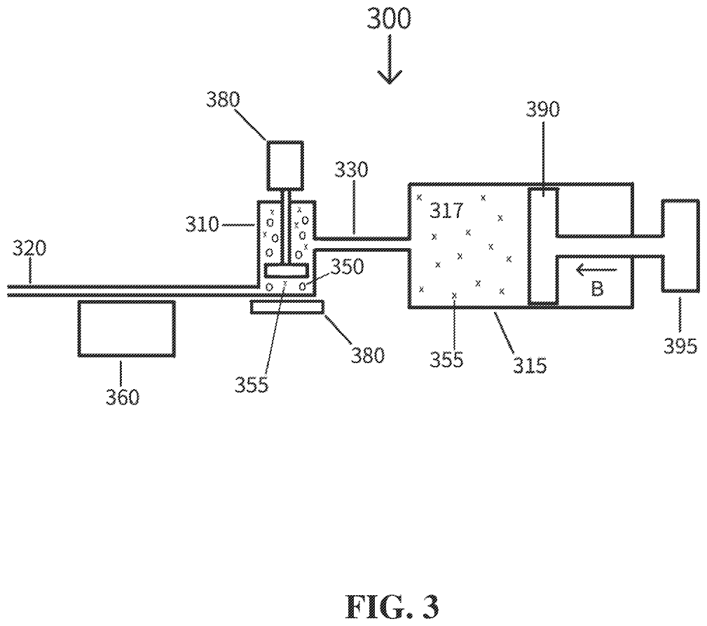

In a separate embodiment shown in FIG. 3, apparatus 300 may include a mixing chamber reservoir 310 and a mixing device and an agitation mechanism 380 configured to agitate cell suspension 350. In this embodiment, first reservoir 315 carries a liquid 317 that is compatible with the cell suspension 350 in the second reservoir 310, and an agitation mechanism 380 is located the second reservoir 310. In the embodiment shown, apparatus 300 comprises liquid transfer mechanism 390, which can be configured as any device capable of transferring liquid 317 from first reservoir 315 to second reservoir 310 via conduit 330. In one specific embodiment, liquid transfer mechanism 390 may be configured as a syringe actuated by a syringe pump 395.

During agitation or mixing of reservoir 310, the forward movement (in the direction of arrow B) of liquid 317 from the 315 reservoir is initiated by liquid transfer mechanism 390, creating displacement of the cell suspension 350 from the 310 reservoir and into the cannula 320. The agitation mechanism 380 may include a motor-driven stirring device, or a shaking device, or a rocking device, or rotating magnetic device, analogous to agitation mechanisms 180 and 280.

In certain embodiments, monitoring devices (160, 260 or 360) may comprise a sensor that can detect a parameter that can be correlated to the concentration of cells. For example, the monitoring device may comprise an optical sensor that detects an amount of light transmitted through (or reflected or absorbed by) cell suspension 150, 250, or 350 to determine the concentration of cells. In other embodiments, the monitoring device may comprise a flow sensor capable of measuring the turbidity of cell suspension or detecting macromolecules, including for example, sensors available from Pendotech© or PreciGenome©. The monitoring device may also be configured (e.g. via a computer processor and a computer readable medium) to quantify the number of cells moving transferred to cannula 120, 220 or 320.

Compared to a stationary reservoir, the advantages offered to the arrangements shown in FIGS. 1-3 are that cells remain in suspension and are less likely to settle out of suspension prior to injection. Agitation mechanisms 180, 280, and 380 are mechanisms that provide the energy necessary to prevent cells from settling out of suspension.

For example, by locating agitation mechanism 280 and second reservoir 210 proximal to cannula 232, cell suspension 250 does not have to travel from proximal end 231 of conduit 230 prior to entering cannula 232. Accordingly, there may be less time between when cell suspension 250 is agitated via agitation mechanism 280 and when cell suspension 250 is injected via cannula.

In other embodiments, the arrangements shown in FIGS. 1-3 would include supplementation of the suspension medium 255 with soluble factors (molecules) that increase the viscosity of the cell suspension medium 255 and thereby increase the settling time of the cells. One such soluble factor is hyaluronic acid (hyaluronan, or HA).

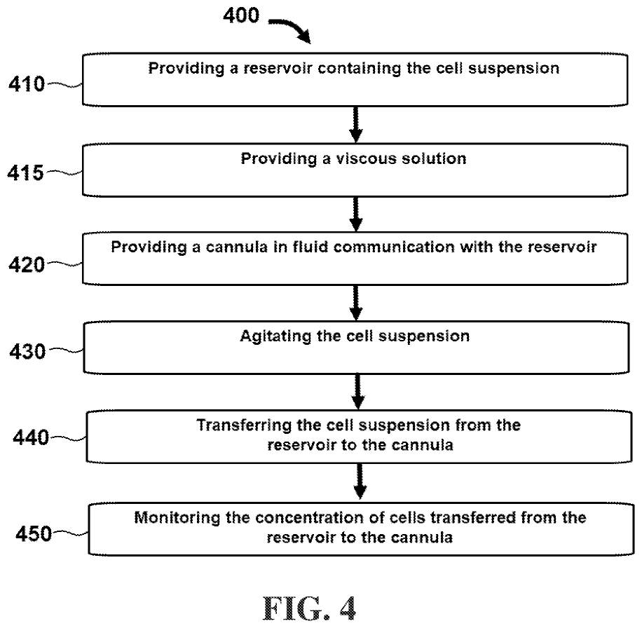

Referring now to FIG. 4, a flowchart 400 provides steps that can be used in a method for monitoring and delivering a cell suspension. As shown in FIG. 4, step 410 includes providing a reservoir containing a cell suspension. Step 415 includes providing a viscous solution. Step 420 recites providing a cannula in fluid communication with the reservoir, while step 430 discloses agitating the cell suspension. Step 440 includes transferring the cell suspension from the reservoir to the cannula, and step 450 recites monitoring the concentration of cells transferred from the reservoir to the cannula.

### EXAMPLES

The following examples are included to demonstrate exemplary embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

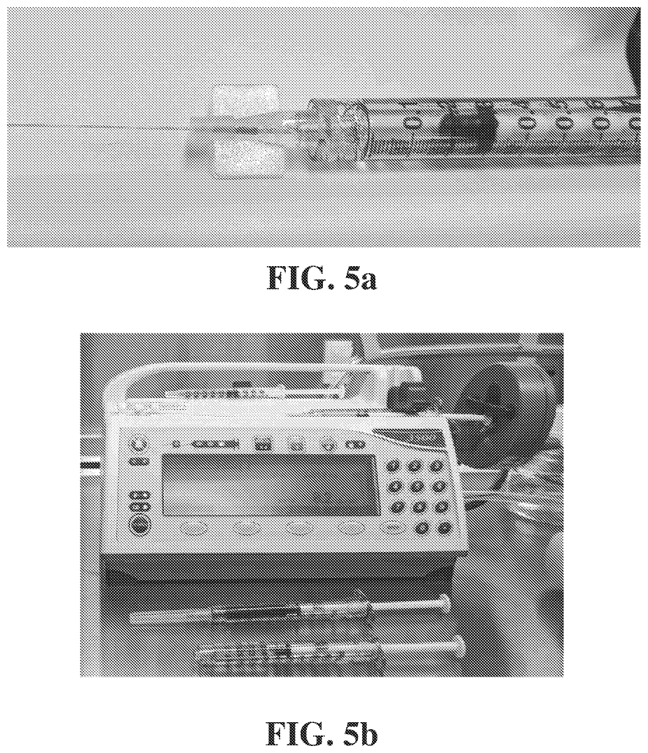

The example shown in FIG. 5 shows an apparatus that allows controlled transfer of highly concentrated cell suspensions from a syringe into medical tubing, and at the same time monitoring the concentration (or dose) of cells delivered through such a tubing. In this example, the apparatus can achieve controlled transfer by mechanical rocking, vibration, or rotation in such a way as to create turbulence within a syringe. This example includes a syringe loaded with a certain volume of cells and also an air bubble or an object that moves within the syringe creating turbulence. In some examples, the mechanical instrument may be a rocking platform or a servo motor or a shaking device.

In this example, the component part of the device that allows monitoring cell concentrations is a sterile glass fiber tubing that allows the passage of incident light. In addition, the proximal end of the tubing will be embedded into a Luer Lock fitting that will provide an attachment point for a syringe. Near the proximal end, the tubing will be embedded into, or pass through, a flow cell chamber comprised of a transparent glass platform (e.g., a glass slide). This platform would serve as a stage on which to focus the incident light. Light transmitted through the tubing would pass to an objective microscope lens and then to an imaging sensor (CCD). The objective lens would have sufficient magnification to resolve single cells passing through the flow cell.

Certain embodiments can include the iPS Cube device, which is a custom-made flow cell that is an example of such a configuration. Thus, this embodiment could include an iPS Cube flow cell integrated into the tubing of the MRI Interventions SmartFlow® Neurocannula system. The iPS Cube could be retrofitted with a higher power objective lens for better resolution of single cells, and the software component of this system could include tools to quantify the number of cells moving through a defined length and volume of tubing, and to thus determine the concentration of cells loaded into the tubing. In this manner, the transfer of cells (the dose) from a holding vessel (the syringe or test tube) to the tubing will be controlled and quantifiable.

In a separate embodiment, the transparent unprotected SmartFlow tubing will pass through a flow sensor that is capable of turbidity measurements, or the detection of macromolecules. Pendotech® is a supplier of such monitoring devices. Similar to optical imaging, incident light of a defined wavelength would be passed through the tubing to a detector. However, rather than using image analysis software, photometric changes in absorbance would be correlated to the concentration of cells. For example, absorbance of 800 nm light could be used to monitor turbidity. Ultraviolet (UV) light absorbance could be used to monitor nucleic acid concentrations. Either or both measurements may be useful correlates of cell concentration. Such a device is expected to have significant value for surgical trials requiring controlled dosing of cells; particularly those involving cell delivery to organs and solid tissue.

Embodiments of the invention could be useful for a cell therapy product that requires transfer of high concentrations of cells into medical tubing for cell delivery, including for example, those for the treatment of Parkinson's disease.

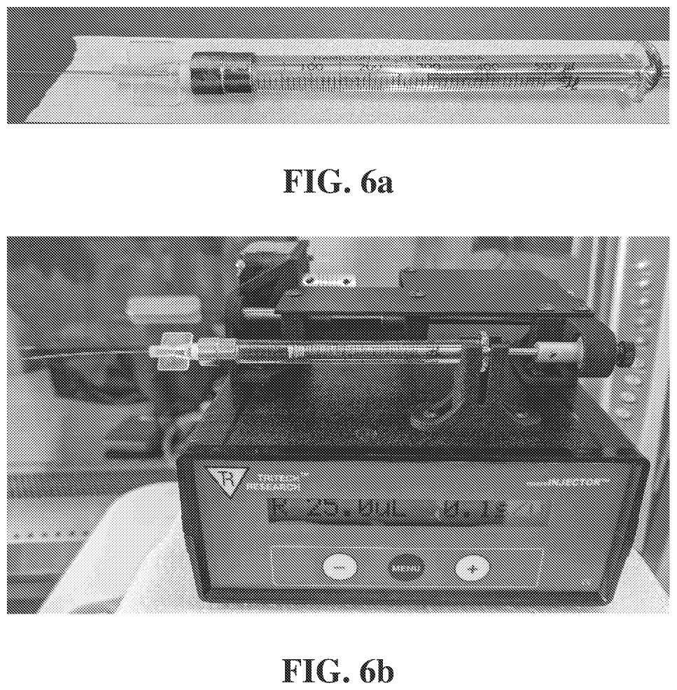

There are several commercially available syringe pumps that are sold for controlled delivery of small volumes suitable for use in exemplary embodiments. These devices typically employ a motor-driven mechanical arm that pushes the syringe plunger. The syringe is meant to be held in a fixed horizontal or vertical position during operation of the pump. Testing has been conducted on two Syringe pumps: the Medfusion® 3500 (Smiths-Medical) shown in FIG. 5b, and the microInjector™ MINJ-PD (Tritech Research) shown in FIG. 6b.

Because the cell product (in standard saline solutions) undergoes a phase separation within a few minutes, cell settling occurs before the time required to assemble the device. The typical steps are: (1) attach the syringe to the medical tubing and neuro-cannula (avoiding air bubbles); (2) mount the syringe on the pump, and (3) program the unit to deliver a defined volume at a defined flow rate. Assembly time is required for both the Medfusion® 3500 pump (with a 1 ml disposable syringe), as well as the microInjector™ Tritech pump.

The microInjector™ Tritech pump accommodates a smaller volume (Hamilton®) syringe which should offer better precision of delivered small volumes. Furthermore, unlike most other syringe pumps, the microInjector™ Tritech pump has the capability to move the plunger bi-directionally and thus agitate the contents of the syringe. However, in testing, the latter feature has proven to be ineffective at keeping cells in suspension.

One solution considered is to continuously mix the cells within the syringe during cell delivery. This could be accomplished by mounting the syringe to the pump and then: (1) placing the entire pump on a rocking platform; or (2) coupling the pump body to a servo motor capable of rotating the pump through 180° oscillations. Conceptually, both ideas may serve to prevent cell settling by using gravity to move the cells within the syringe. However, in practice, gravity alone has proven an insufficient mixing force. This led to additional strategies being considered to create turbulence within the syringe during mixing.

Another embodiment may include a magnetic ball bearing in the syringe barrel and inducing it to roll along the length of the barrel by rocking or rotating the device. Alternatively, the ball bearing could be moved bi-directionally by a motorized magnetic arm moving along the outside of the syringe. In either scenario, the movement of the ball bearing would be optimized to create enough turbulence to prevent cells from settling.

In another scenario, a magnet could be rotated around the syringe barrel; for example, this commercially available device utilizes a belt-driven rotating magnet and a stirring magnet within the syringe. The design of the latter device could be adapted for use with cell suspensions.

Certain embodiments may utilize a non-metallic bead (e.g. plastic), that could be more or less dense than aqueous solution, such that the non-metallic bead could sink or float. Other embodiments could utilize the introduction of an air bubble as a substitute for the ball bearing. The rocking platform or the rotating motion of a servo motor could be tuned to the movement of the air bubble within the barrel of the syringe in order to optimize the turbulence created within the cell suspension, and prevent cell settling. Furthermore, the mixing device could be momentarily stopped with the syringe in a vertical orientation (pointing down), and the air bubble at the top of the syringe (between the plunger and the cell suspension) to minimize the risk that air would exit the syringe (and enter the patient) during operation of the syringe pump and delivery of a small volume into the tubing. Preliminary evidence indicates that the air bubble mixing strategy is effective at keeping high concentrations of cells in solution. This strategy improves the consistency of cell delivery (the number of cells delivered through medical tubing) in laboratory testing, and predicts better accuracy in dosing in a clinical setting

In certain embodiments, the conduit may include Smart Flow® tubing which is part of MRI Interventions ClearPoint® Neuro Navigation cannula product

Briefly, this product is composed of narrow glass tubing that has a polymer coating. At the proximal end, the tubing can be connected to a syringe with a Luer Lock fitting. The opposite (distal) end of the tubing is encased by a ceramic cannula which is rigid enough for precise insertion into the brain—this is the working end of the product. The tip of the cannula is narrow and intended to penetrate the brain tissue with minimal damage. The overall length of the tubing is customizable, and determines how large of a volume of cells might be held.

In one scenario for loading cells into the SmartFlow® tubing, cells are mixed thoroughly in a standard conical (Eppendorf) tube, loaded into a syringe with a LuerLock® port, then the syringe is transferred on to the SmartFlow® tubing, and then the cell suspension is immediately pushed into the tubing manually (e.g. by a human thumb on a plunger). This strategy requires that the cells are loaded into the tubing quickly before they settle out of solution. Subsequently, a syringe pump could be used to deliver small volumes (doses).

In the laboratory setting, this “pre-filling” approach has shown limited success. Consider a four-foot long piece of SmartFlow® tubing with an inner diameter of 200 microns and pre-loaded with cells (as described above). The total volume within this tubing is approximately 40 ul. Thus, the number of accurate doses is limited by the length of the tubing; in this example 16 doses at 2.5 ul might be available (16×2.5=40 μl). After delivery of the pre-loaded 40 ul, one would expect the dosing inaccuracy to return because the remaining cells in the syringe would have undergone settling before displacement of the pre-loaded 40 ul volume.

As an alternative to rapidly loading cells into the proximal end of the tubing, the inventors have also conceived of strategies to “pull” the cells into the distal tip of the SmartFlow® tubing (i.e., the cannula end of the tubing.) Cells could be prepared at high concentrations in a conical tube and mixed continuously using a vortex mixer (such as the Thermo Scientific™ LP Vortex Mixer) or a shaking device. A syringe could be connected to the proximal end of the tubing and mounted onto a syringe pump. The pump could then pull the plunger to apply a controlled suction force which would pull cells into the cannula tip. After pre-filling the tubing with the cell suspension, the cannula tip would be inserted into the (patient's) brain, the pump could be re-programmed to reverse the direction of flow, and doses of cells could be delivered.

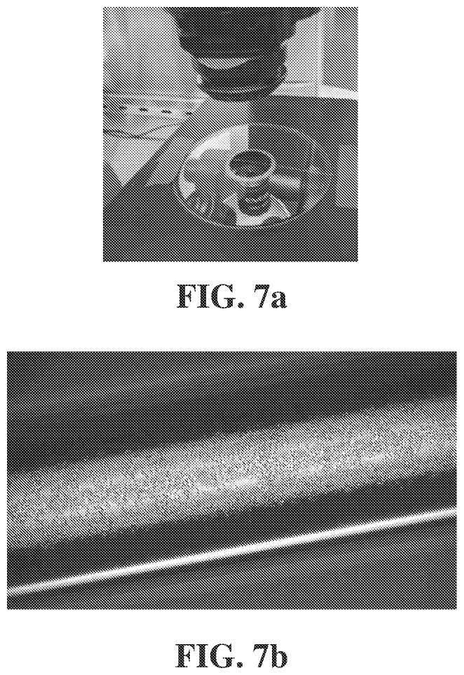

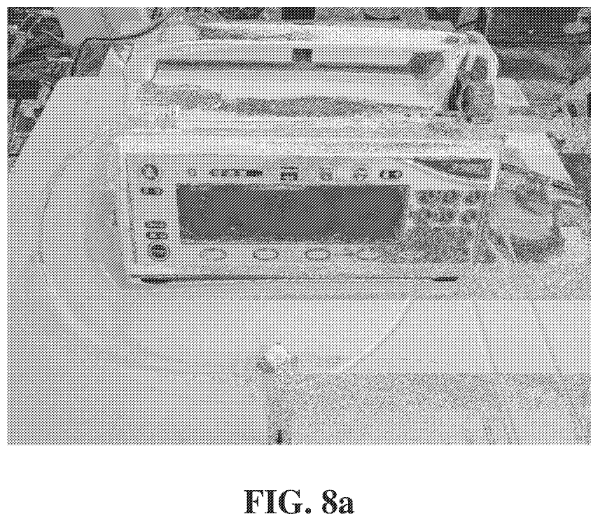

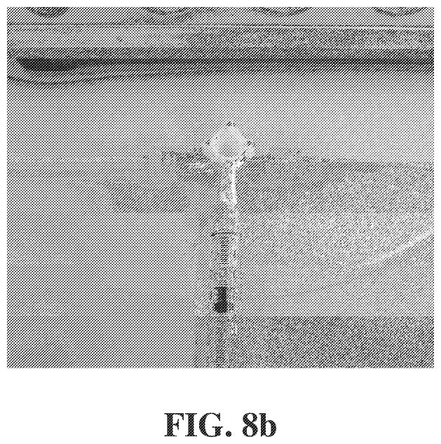

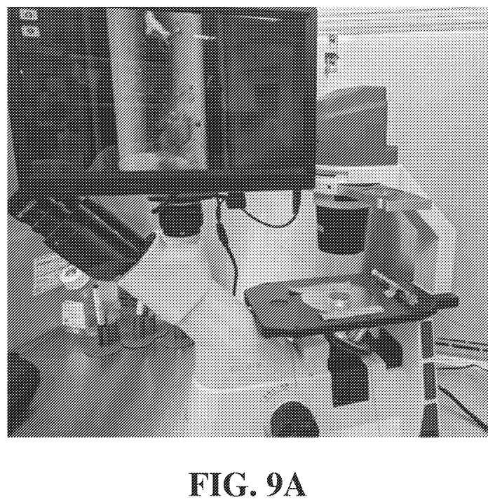

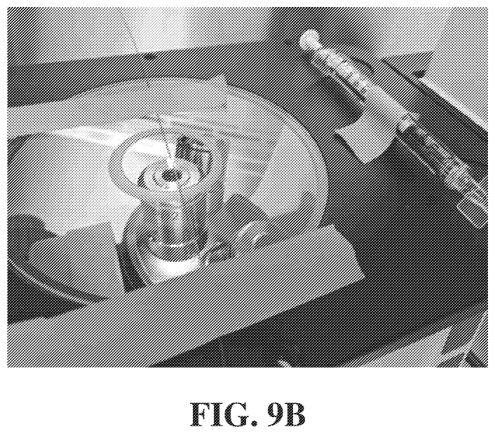

Some of the SmartFlow® product samples that have been tested have been received without the protective polymer coating. These test samples are not 510-compliant and intended only for R&D use. It has been discovered that it is possible to visualize cells moving through this unprotected tubing. This was demonstrated by extending the tubing across the stage of a Leica EXI-310-PH phase contrast microscope coupled with a video camera and monitor FIGS. 9a and 9b. Video and still photo images (such as FIG. 7b) were obtained while pumping cells through the tubing.

This was recognized as a possible method to control dosing in the aforementioned neurosurgery scenario. If an imaging or monitoring device was attached or integrated onto an uncoated section of the four-foot long tubing—in between the syringe (at the proximal end of the tubing) and the cannula tip (at the distal end)—and that detection device was capable of quantifying the concentration of cells passing by it, then it would be possible to create a cell delivery device with an in-line dosing control feature. The detection device would report any differences in the concentration of the cells in the syringe compared to concentration of cells moving through the tubing toward the cannula tip. In a clinical setting, this would enable the neurosurgeon to have a much better understanding of how many cells are being delivered to the patient, i.e., how accurate is the dosing for repeated small volume deposits.

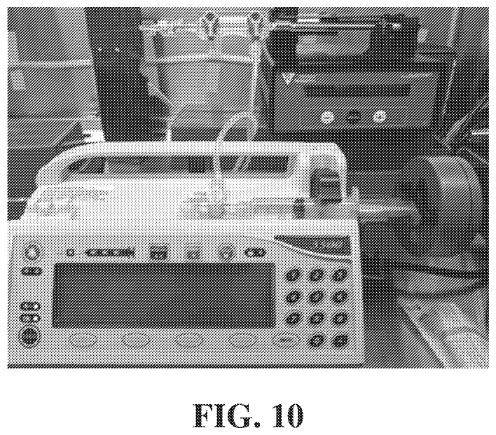

Referring now to FIG. 2, a schematic of an apparatus that uses a hybrid approach. This embodiment uses a hybrid approach with both a Medfusion® 3500 pump and the microInjector® Tritech pump. In operation, a user can fill a syringe mounted onto Medfusion® 3500 pump, and push BSS to fill tubing and valves via a first port. The user can also fill the tubing from the cannula tip with cell suspension, using the 250 ul Hamilton® syringe on the microInjector™ Tritech pump (in reverse). A second port can be used to bleed out air bubbles as shown in FIG. 10.

Increased Viscosity Example

In a separate embodiment, to minimize the speed of sedimentation, the physiologic saline carrier solution will be supplemented with soluble compounds that increase the viscosity of the cell suspension.

The mathematical relationship (Stokes' Law) that describes velocity of sedimentation of a sphere falling in a fluid is given below:

$$v = \frac{2}{ℊ} \frac{( {ρ}_{p} - {ρ}_{f} )}{μ} ℊ {R}^{2}$$

Where:
g is the gravitational field strength (m/s²)
R is the radius of the spherical particle (m)
ρₚ is the mass density of individual cells (kg/m³)
ρ_{f} is the mass density of the fluid (kg/m³)
μ is the viscosity (kg/(m*s)).

Using the calculation above, the theoretical sedimentation rate of individual cells in physiologic saline solutions occurs at approximately 0.35 mm/minute. However, in practice we have observed significantly higher sedimentation rates. This is likely explained by the affinity that dopaminergic progenitor cells (DAPC) have to each other. High density suspensions of DAPC form clusters of cells, effectively increasing the radius (R) value in the above equation, resulting in significantly faster rates of sedimentation. This phenomenon has been visualized in real time with microscopic imaging.

Hyaluronon, or hyaluronic acid (HA) is a high molecular weight polysaccharide that is abundant in the extracellular spaces of the brain. This compound has been utilized in clinical ophthalmology surgeries, as well as cosmetic applications into the skin. It is available in several formulations (such as Healon®) that have viscosities that are several orders of magnitude greater than physiologic saline solutions. HA also has several desirable characteristics that make it suitable for delivery to the brain. It is biodegradable, biocompatible, non-toxic, and non-immunogenic. See U.S. Pat. No. 4,141,973; Trombino et al., “Strategies for Hyaluronic Acid-Based Hydrogel Design in Drug Delivery”; Pharmaceutics 2019, 11, 407. Richter, W., Ryde, M. & Zetterström, O.: Nonimmunogenicity of a purified sodium hyaluronate preparation in man. Int Arch Appl Immun 59:45-48 (1979).; Richter, W.: Non-immunogenicity of purified hyaluronic acid preparations tested by passive cutaneous anaphylaxis. Int Arch All 47 (1974) p 211-217.

By adding a supplement to the saline solution that both increases the viscosity and decreases the clumping phenomenon, it is possible to slow the sedimentation kinetics. This is expected to translate into more practical applications of the medical devices described within this application. HA solutions that impart varying amounts of viscosity will be studied to select a concentration and molecular composition of HA that provides the most consistent transfer of a cell suspension from a container (an Eppendorf tube, or a syringe) into the medical tubing over time.

Preliminary evidence using a suspension of DAPC in BSS Plus supplemented with 0.1% Healon GV® showed significantly slower sedimentation kinetics, as well as less cell clumping. Using video imaging, we have also observed the transfer of DAPC suspensions from tubes and syringes into the SmartFlow medical tubing, and have shown that the presence of HA significantly mitigated the clumping phenomenon.

Importantly, the use of HA, or similar biocompatible molecules that increase viscosity, are expected to greatly extend the time available to prepare DAPC, transfer them to the medical tubing, and finally transfer cells into the PD patient with more precise control over the number of cells delivered. In other words, the reduced sedimentation rate is expected to translate into more consistent dosing for this type of cell therapy.

All of the devices, apparatus, systems and/or methods disclosed and claimed herein can be made and executed without undue experimentation in light of the present disclosure. While the devices, apparatus, systems and methods of this invention have been described in terms of particular embodiments, it will be apparent to those of skill in the art that variations may be applied to the devices, apparatus, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
RA Barker, Lancet Neurol. 2013 January; 12(1) 84-91.
MH Amer, et. al. Nature Partner Journals Regenerative Medicine, (2017).
https://blogs.rsc.org/chipsandtips/2007/08/21/preventing-suspension-settling-during-injection/
https://www.cetoni.com/products/syringe-stirrer-nemix/
https://www.mriinterventions.com/rnoducts/mri-guided-drug-delivery/smartflow-product-sheet
U.S. Pat. No. 4,141,973
Trombino, S.; Servidio, C.; Curcio. F.; Cassano R.; Strategies for Hyaluronic Acid-Based Hydrogel Design in Drug Delivery. Pharmaceutics 2019, 11, 407.
Seliktar, D.; Designing Cell-Compatible Hydrogels for Biomedical Applications. 2012 Jun. 1; 336(6085):1124-8. doi: 10.1126/science.1214804.
Richter, W., Ryde, M. & Zetterström. O.: Nonimmunogenicity of a purified sodium hyaluronate preparation in man. Int Arch Appl Immun 59:45-48 (1979).
Richter, W.: Non-immunogenicity of purified hyaluronic acid preparations tested by passive cutaneous anaphylaxis. Int Arch All 47 (1974) p 211-217.

1. An apparatus for monitoring and delivering a cell suspension, the apparatus comprising:
a reservoir containing the cell suspension;
a cannula in fluid communication with the reservoir;
a liquid transfer mechanism configured to transfer the cell suspension from the reservoir to the cannula;
an agitation mechanism configured to agitate the cell suspension; and
a monitoring device configured to quantify a concentration of cells transferred from the reservoir to the cannula.

2. The apparatus of claim 1 wherein the monitoring device records a digital image of the cell suspension.

3. The apparatus of claim 2 wherein the digital image is a still image.

4. The apparatus of claim 2 wherein the digital image is a moving image.

5. The apparatus of claim 1 wherein the monitoring device is configured to quantify a number of cells transferred to the cannula.

6. The apparatus of claim 1 wherein the monitoring device is an optical measurement device.

7. The apparatus of claim 6 wherein the optical measurement device is configured to detect an amount of light transmitted through the cell suspension.

8. The apparatus of claim 6 wherein the optical measurement device is configured to detect an amount of light transmitted reflected by the cell suspension.

9. The apparatus of claim 6 wherein the monitoring device is configured to measure the turbidity of the cell suspension.

10. The apparatus of claim 1 wherein the liquid transfer mechanism comprises a syringe.

11. The apparatus of claim 10 wherein the syringe is a threaded syringe.

12. The apparatus of claim 10 wherein the liquid transfer mechanism comprises a syringe pump.

13. The apparatus of claim 1 wherein the liquid transfer mechanism comprises a pump.

14. The apparatus of claim 13 wherein the pump is a peristaltic pump.

15. The apparatus of claim 1 wherein the liquid transfer mechanism comprises an inflatable bladder.

16. The apparatus of claim 1 wherein the apparatus further comprises a conduit in fluid communication with the reservoir and the cannula.

17. The apparatus of claim 16 wherein:
the conduit comprises a proximal end and a distal end;
the reservoir is coupled to the proximal end of the conduit; and
the cannula is coupled to the distal end of the conduit.

18. The apparatus of claim 1 wherein the agitation mechanism is configured to impart motion to the reservoir.

19. The apparatus of claim 1 further comprising a mixing element in the reservoir.

20. The apparatus of claim 19 wherein the mixing element is a bubble.

21. The apparatus of claim 19 wherein the mixing element is a spherical ball bearing.

22. The apparatus of claim 19 wherein the mixing element is a bead.

23. An apparatus for monitoring and delivering a cell suspension, the apparatus comprising:
a first reservoir containing a liquid;
a second reservoir containing a cell suspension;
an agitation mechanism configured to agitate the cell suspension;
a conduit in fluid communication with the first reservoir and the second reservoir;
a cannula in fluid communication with the conduit and the second reservoir; and
a liquid transfer mechanism configured to transfer the cell suspension from the second reservoir to the cannula.

24. The apparatus of claim 1 wherein the monitoring device is configured to quantify a number of cells transferred to the cannula.

25. The apparatus of claim 23 wherein:
the liquid transfer mechanism is configured to move in a first direction to transfer the cell suspension from the second reservoir to the cannula; and
the liquid transfer mechanism is configured to move in a second direction to transfer the cell suspension through the cannula.

26. The apparatus of claim 23 wherein:
the conduit comprises a proximal end and a distal end;
the first reservoir is coupled to the proximal end of the conduit; and
the second reservoir is coupled to the distal end of the conduit.

27. The apparatus of claim 23 wherein the monitoring device is an optical measurement device.

28. The apparatus of claim 27 wherein the optical measurement device is configured to detect an amount of light transmitted through the cell suspension.

29. The apparatus of claim 27 wherein the optical measurement device is configured to detect an amount of light transmitted reflected by the cell suspension.

30. The apparatus of claim 27 wherein the monitoring device is configured to measure the turbidity of the cell suspension.

31. The apparatus of claim 23 wherein the liquid transfer mechanism comprises a syringe.

32. The apparatus of claim 23 wherein the syringe is a threaded syringe.

33. The apparatus of claim 31 wherein the liquid transfer mechanism comprises a syringe pump.

34. The apparatus of claim 23 wherein the liquid transfer mechanism comprises a pump.

35. The apparatus of claim 34 wherein the pump is a peristaltic pump.

36. The apparatus of claim 23 wherein the liquid transfer mechanism comprises an inflatable bladder.

37. The apparatus of claim 23 wherein the apparatus further comprises a conduit in fluid communication with the reservoir and the cannula.

38. The apparatus of claim 37 wherein:
the conduit comprises a proximal end and a distal end;
the reservoir is coupled to the proximal end of the conduit; and
the cannula is coupled to the distal end of the conduit.

39. The apparatus of claim 23 wherein the agitation mechanism is configured to impart motion to the reservoir.

40. The apparatus of claim 23 further comprising a mixing element in the reservoir.

41. The apparatus of claim 40 wherein the mixing element is a bubble.

42. The apparatus of claim 40 wherein the mixing element is a spherical ball bearing.

43. A method for monitoring and delivering a cell suspension, the method comprising:
providing a reservoir containing the cell suspension;
providing a cannula in fluid communication with the reservoir;
agitating the cell suspension;
transferring the cell suspension from the reservoir to the cannula; and
monitoring a concentration of cells in the cell suspension transferred from the reservoir to the cannula.

44. The method of claim 43 wherein transferring the cell suspension from the reservoir to the cannula comprises transferring the cell suspension through a conduit.

45. The method of claim 43 wherein monitoring the concentration of cells transferred from the reservoir to the cannula comprises measuring an optical quality of the cell suspension.

46. The method of claim 43 wherein:
the reservoir containing the cell suspension is a first reservoir; and
the method further comprises:
transferring a liquid from a second reservoir to the cannula; and
diluting the cell suspension with the liquid.
